(19) United States
(12) Patent Application Publication
(10) Pub. No.: US 2011/0257684 A1
(43) Pub. Date: Oct. 20, 2011

(54) ALA RODS FOR LUMBAR-SACRAL INTERSPINOUS PROCESS DEVICE

(75) Inventor: Meera L. Sankaran, Cupertino, CA (US)

(73) Assignee: KYPHON S?RL, Neuchatel (CH)

(21) Appl. No.: 12/763,368

(22) Filed: Apr. 20, 2010

(51) Int. Cl. A61B 17/70; A61B 17/88

(52) U.S. Cl. 606/249; 606/264; 606/279

(57) ABSTRACT

Medical devices for the treatment of spinal conditions are described herein. The medical device includes a main body portion and a mounting portion that is adapted to be fixed with respect to the sacral ala and the main body portion is adapted to support an implant such as an interspinous process spacer. This arrangement maintains distraction between the adjacent spinous processes of the L5 vertebra and the S1 vertebra when the spine moves to extension.

### BACKGROUND

This invention relates generally to the treatment of spinal conditions, and more particularly, to the treatment of spinal stenosis using devices for implantation between adjacent spinous processes.

The clinical syndrome of neurogenic intermittent claudication due to lumbar spinal stenosis is a frequent source of pain in the lower back and extremities, leading to impaired walking, and causing other forms of disability in the elderly. Although the incidence and prevalence of symptomatic lumbar spinal stenosis have not been established, this condition is the most frequent indication of spinal surgery in patients older than 65 years of age.

Lumbar spinal stenosis is a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop lumbar spinal stenosis each year. For patients who seek the aid of a physician for back pain, approximately 12%-15% are diagnosed as having lumbar spinal stenosis.

Common treatments for lumbar spinal stenosis include physical therapy (including changes in posture), medication, and occasionally surgery. Changes in posture and physical therapy may be effective in flexing the spine to decompress and enlarge the space available to the spinal cord and nerves—thus relieving pressure on pinched nerves. Medications such as NSAIDS and other anti-inflammatory medications are often used to alleviate pain, although they are not typically effective at addressing spinal compression, which is the cause of the pain.

Surgical treatments are more aggressive than medication or physical therapy, and in appropriate cases surgery may be the best way to achieve lessening of the symptoms of lumbar spinal stenosis. The principal goal of surgery is to decompress the central spinal canal and the neural foramina, creating more space and eliminating pressure on the spinal nerve roots. The most common surgery for treatment of lumbar spinal stenosis is direct decompression via a laminectomy and partial facetectomy. In this procedure, the patient is given a general anesthesia as an incision is made in the patient to access the spine. The lamina of one or more vertebrae is removed to create more space for the nerves. The intervertebral disc may also be removed, and the adjacent vertebrae may be fused to strengthen the unstable segments. The success rate of decompressive laminectomy has been reported to be in excess of 65%. A significant reduction of the symptoms of lumbar spinal stenosis is also achieved in many of these cases.

Alternatively, the vertebrae can be distracted and an interspinous process device implanted between adjacent spinous processes of the vertebrae to maintain the desired separation between the vertebral segments. Such interspinous process devices typically work for their intended purposes. However, because of the human anatomy some of these devices are not readily usable between certain vertebrae.

The spine is divided into regions that include the cervical, thoracic, lumbar, and sacrococcygeal regions. The cervical region includes the top seven vertebrae identified as C1-C7. The thoracic region includes the next twelve vertebrae identified as T1-T2. The lumbar region includes five vertebrae L1-L5. The sacrococcygeal region includes nine fused vertebrae comprising the sacrum comprising S1-S5 vertebrae and four or five rudimentary members that form the coccyx.

The sacrum is shaped like an inverted triangle with the base at the top. The sacrum acts as a wedge between the two iliac bones of the pelvis and transmits the axial loading forces of the spine to the pelvis and lower extremities. The sacrum is rotated anteriorly with the superior endplate of the first sacral vertebrae angled from about 30 degrees to about 60 degrees in the horizontal plane. The S1 vertebrae includes a spinous process aligned along a ridge called the medial sacral crest. However, the spinous process on the S1 vertebrae may not be well defined, or may be non-existent, and therefore may not be adequate for supporting an interspinous process device positioned between the L5 and S1 spinous processes.

Thus, a need exists for a mechanism that will allow an interspinous process device to be readily positioned between the L5 and S1 spinous processes so that the L5 and S1 vertebrae can be distracted and the interspinous process device can maintain the desired separation between the vertebral segments.

### SUMMARY

An ala rod assembly is described herein that includes a main body portion and a mounting portion extending from an end of the main body portion. The mounting portion may be threaded or otherwise include a rough outer surface to facilitate the insertion and retention of the mounting portion in the bone of ala. In addition, a standard bone void filler may be used and located between the mounting portion and the bone to ensure that the mounting portion, and thus the ala rod assembly, is fixed to the sacral ala. The main body portion extends generally medially from the portion of the ala where the mounting portion is fixed. Thus, with the mounting portion appropriately mounted to the ala, the distal end portion of the main body portion of the ala rod assembly extends adjacent to the medial sacral crest and in close proximity to the spinous process of the immediately superior, i.e. L5, vertebra. The distal end portion of the main body portion of the ala rod assembly thus serves as a support for an interspinous process spacer that is disposed on the upper portion of the distal portion of the ala rod assembly beneath the spinous process of the immediately superior vertebra. Since the ala rod assembly is fixed to the sacrum and functions as an artificial spinous process, the interspinous process spacer can provide adequate distraction between the S1 and L5 vertebrae.

A second ala rod assembly may also be used and located on the opposite side of the medial sacral crest with the main body portion of the second ala rod assembly also extending medially from the mounting portion so the distal end portion of the main body portion is adjacent to the medial sacral crest and the main body portion of the other ala rod assembly. This will place the main body portion of the second ala rod assembly in close proximity to the spinous process of the immediately superior vertebra. If a second ala rod assembly is used, both assemblies should be arranged so that the distal end portions are located adjacent to each other. This will allow these portions of the ala rod assemblies to function as an artificial spinous process to support an interspinous process device that can distract the S1 and L5 vertebrae.

### BRIEF DESCRIPTION OF THE DRAWINGS

FIG. 1 is a rear perspective view of one embodiment of an ala rod assembly with an interspinous process device and a portion of a spine on which they are located;

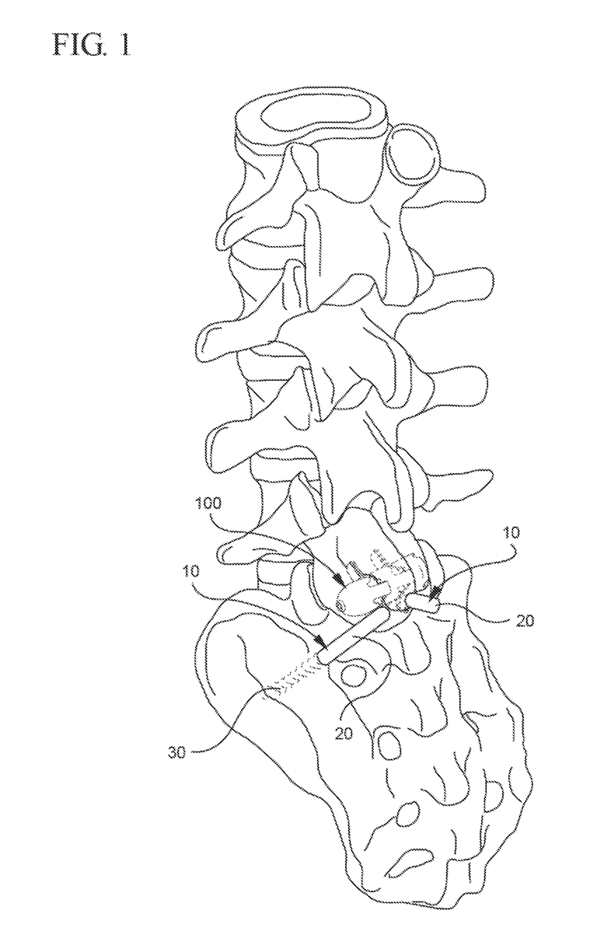

FIG. 2 is a rear elevation view of a portion of a spine and the ala rod assembly and interspinous process device shown in FIG. 1; and

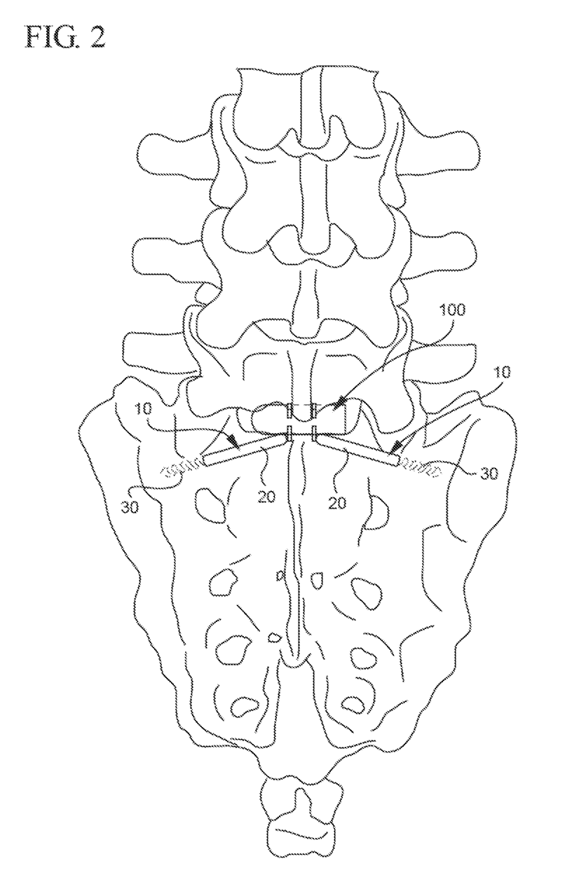

FIG. 3 is a side elevation view of a portion of a spine and the ala rod assembly and interspinous process device shown in FIG. 1.

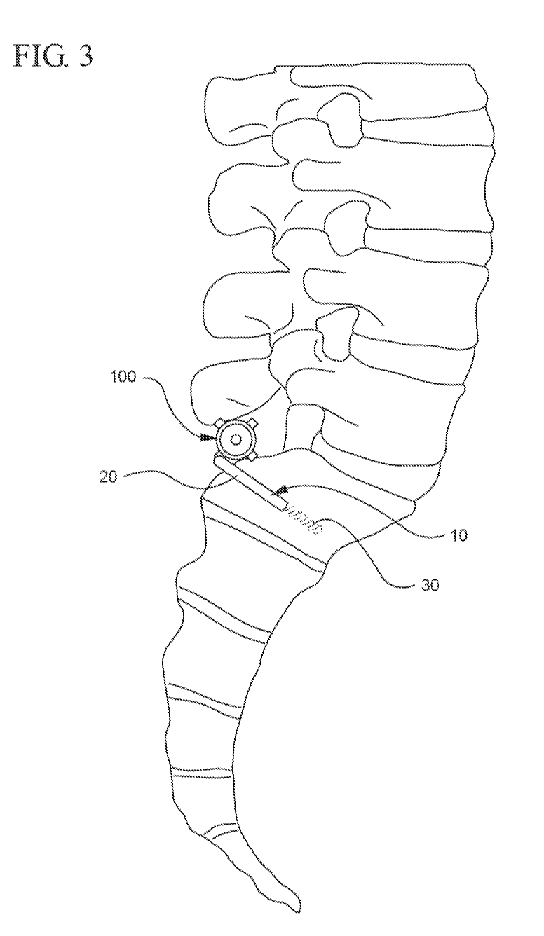

### DETAILED DESCRIPTION

As used in this specification and the appended claims, the singular forms “a,” “an” and “the” include plural referents unless the context clearly dictates otherwise. Thus, for example, the term “a member” is intended to mean a single member or a combination of members, and “a material” is intended to mean one or more materials, or a combination thereof. Furthermore, the words “proximal” and “distal” refer to directions closer to and away from, respectively, an operator (e.g., surgeon, physician, nurse, technician, etc.) who would insert the medical device into the patient, with the tip-end (i.e., distal end) of the device inserted inside a patient's body first. Thus, for example, the device end first inserted inside the patient's body would be the distal end of the device, while the device end last to enter the patient's body would be the proximal end of the device.

As used in this specification and the appended claims, the term “body” when used in connection with the location where the device of this invention is to be placed to treat lumbar spinal stenosis, or to teach or practice implantation methods for the device, means a mammalian body. For example, a body can be a patient's body, or a cadaver, or a portion of a patient's body or a portion of a cadaver.

As used in this specification and the appended claims, the term “parallel” describes a relationship, given normal manufacturing or measurement or similar tolerances, between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

As used in this specification and the appended claims, the terms “normal”, perpendicular” and “orthogonal” describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal, perpendicular or orthogonal to a curved surface when the line and the curved surface intersect at an angle of approximately 90 degrees within a plane. Two geometric constructions are described herein as being “normal”, “perpendicular”, “orthogonal” or “substantially normal”, “substantially perpendicular”, “substantially orthogonal” to each other when they are nominally 90 degrees to each other, such as for example, when they are 90 degrees to each other within a tolerance. Such tolerances can include, for example, manufacturing tolerances, measurement tolerances or the like.

The ala rod assembly is designed to be affixed to the sacral ala. This portion of the sacrum lies in an area away from the cauda equina nerve roots and is a relatively safe area in which to affix the assembly. In addition, this portion of the sacrum provides enough support for the ala rod assembly to allow the assembly to support an interspinous process spacer to allow distraction between the L5 and S1 vertebrae.

An ala rod assembly 10 as described herein includes a main body portion 20 and a mounting portion 30 extending from an end of main body portion 20. One or two such rod assemblies 10 may be used. The end of main body portion 20 may include some feature that will interface with a driver that the surgeon may use to insert rod assembly 10 into sacrum. For example, the end of main body portion 20 may include a flat surface that interfaces with a mating driver. As shown in the figures, one rod assembly 10 is located on one side of the medial sacral crest and a second rod assembly 10 is also used and located on the opposite side of the medial sacral crest. It is to be understood that the configuration of the two rod assemblies are substantially identical but with the rod assemblies extending medially from the portion of the ala to which they are mounted and oriented in mirror-image arrangements on either side of the medial sacral crest. The description of rod assembly 10 that follows is directed to a single such assembly with the understanding that the other rod assembly has substantially the same configuration.

Mounting portion 30 of rod assembly 10 may be threaded. Alternatively it may include a textured outer surface to allow mounting portion 30 to be inserted into the bone of ala. The textured outer surface may include points or troughs that allow for better “bite” into the bone. In addition, a standard bone void filler may be applied between mounting portion 30 and the bone to ensure that mounting portion 30, and thus rod assembly 10, is fixed to the sacral ala.

Main body portion 20 extends generally longitudinally from mounting portion 30 so as to have a distal end portion adjacent to the medial sacral crest and in close proximity to the spinous process of the immediately superior, i.e. L5, vertebra when mounting portion 30 is fixed to the sacral ala at an appropriate angle. If desired, main body portion 20 may extend at an angle to mounting portion 30, although such a configuration makes a minimally invasive implantation method more challenging. Rod assembly 10 should be implanted such that main body portion 20 is generally parallel to the spinous process of the immediately superior vertebra. Alternatively, rod assembly 10 may be implanted so that main body portion 20 forms an acute angle with the spinous process of the immediately superior vertebra. Such an acute angle will ensure that any spacer located on rod assembly 10 will be under compression between rod assembly 10 and the spinous process of the immediately superior vertebra. The specific angle between main body portion 20 of rod assembly 10 and the immediately superior spinous process may depend on the specific spacer used in connection with rod assembly 10.

In addition, a portion of main body portion 20 extends posteriorly beyond the sacral spinous process. The distal end portion of rod assembly 10 thus acts as an artificial spinous process having a sufficient superior surface area to serve as a support for an implant, such as an interspinous process spacer 100. The superior surface of main body portion 20 may include a cupped or recessed portion to hold spacer 100 in place to prevent it from moving off of rod assembly 10. Alternatively, main body portion 20 may interlock with spacer 100. Regardless of the configuration of the superior surface of main body portion 20, interspinous process spacer 100 should be disposed on the superior surface of main body portion 20 so as to extend to the inferior surface of the spinous process of the immediately superior vertebra. In this manner, interspinous process spacer 100 can provide adequate distraction between the S1 and L5 vertebrae.

As described above, an interspinous process spacer may be supported by rod assembly 10 described herein. Such an implant may be positioned between the L5 spinous process and rod assembly 10 to provide relief for lumbar spinal stenosis. Specifically, an inferior surface of the interspinous process spacer contacts the superior surface of the distal end portion of rod assembly 10 and a superior surface of the interspinous process spacer contacts the L5 spinous process to provide the desired distraction.

Rod assembly 10 may be used in connection with various different interspinous process spacers, such as those shown in more detail in U.S. Patent Application Publication Nos. 2005/0261768, 2007/0225807, 2008/0039859 and 2008/0086212, the entire contents of which are hereby expressly incorporated herein by reference. The foregoing interspinous process spacers are exemplary only and other spacers can also be used with rod assembly 10 described herein. The specific configuration of the interspinous process spacer is not important for purposes of the general description of rod assembly 10 disclosed herein. Of course, some portion of rod assembly 10 may be modified to ensure proper engagement of rod assembly 10 and the interspinous process spacer to ensure proper distraction, but such modifications are contemplated and are within the scope of this invention.

Rod assembly 10 may be implanted in a minimally invasive manner. In addition, rod assembly 10 may be implanted percutaneously. In such a method, a cannula can be inserted into the patient such that the distal opening of the cannula is located adjacent to the location of the sacral ala where rod assembly 10 is to be fixed. If desired, the cannula may be inserted in a medial-to-lateral approach. The surgeon may use fluoroscopy to visualize the location of the distal end of the cannula to ensure proper positioning. Such a method is disclosed in co-pending U.S. patent application Ser. No. 11/778,453 filed Jul. 16, 2007 the entire contents of which are hereby expressly incorporated herein by reference. Rod assembly 10 may then be inserted through the cannula and embedded in the sacral ala. Mounting portion 30 of rod assembly 10 may be oriented distally during insertion. Also, once the surgeon confirms the proper location of mounting portion 30 with respect to the sacral ala, the surgeon may affix mounting portion 30 to the sacral ala. The surgeon may apply a bone void filler between mounting portion 30 and the sacrum if desired. If a second ala rod assembly is to be fixed on the other side of the medial sacral crest, a second cannula may also be inserted into the patient to the other side of the medial crest to allow the surgeon to insert the second rod assembly therethrough. Again, a medial-to-lateral approach may be used. When a second rod assembly is used, the distal portion of the main body portions of each of the rod assemblies should be inserted and affixed to the sacrum such that they are adjacent to each other and aligned adjacent to the sagittal plane and inferior to the spinous process of the L5 vertebra.

The angle at which the cannula engages the ala during the implantation step should be such that when rod assembly 10 is inserted through the cannula and is fixed to the ala, the distal portion of rod assembly 10 is posterior of the sacral spinous process to form an“artificial spinous process” that is larger or extends more posteriorly than the existing S1 spinous process. This provides a superior surface on which an implant, such as an interspinous process spacer, may be located. Where two rod assemblies are used, the distal portions of the two assemblies should intersect or be closest to each other adjacent to and posterior of the sacral spinous process and form the desired robust “artificial spinous process”. Once rod assembly 10 is properly positioned, interspinous process spacer 100 may be inserted percutaneously into the L5-S1 space so spacer 100 is located between the distal portion of rod assembly 10 and the L5 spinous process.

The ala rod assembly described herein can be constructed with various biocompatible materials such as, for example, titanium, titanium alloy, surgical steel, biocompatible metal alloys, stainless steel, Nitinol, plastic, polyetheretherketone (PEEK), carbon fiber, ultra-high molecular weight (UHMW) polyethylene, and other biocompatible polymeric materials.

While various embodiments of the ala rod assembly have been described above, it should be understood that they have been presented by way of example only, and not limitation. Many modifications and variations will be apparent to the practitioner skilled in the art. The foregoing description of the ala rod assembly is not intended to be exhaustive or to limit the scope of the invention. It is intended that the scope of the invention be defined by the following claims and their equivalents.

What is claimed is:

1. A medical device, comprising:
a main body portion having a longitudinal axis, a distal portion and a proximal portion;
a mounting portion having a longitudinal axis, a distal portion and a proximal portion, wherein the distal portion of the mounting portion is adjacent to the proximal portion of the main body portion;
wherein the mounting portion has an outer surface that is adapted to engage bone to fix the mounting portion thereto and the distal portion of the main body portion is adapted to support an interspinous process spacer thereon.

2. The medical device of claim 1 wherein the longitudinal axis of the main body portion is substantially aligned with the longitudinal axis of the mounting portion.

3. The medical device of claim 1 wherein the longitudinal axis of the main body portion intersects the longitudinal axis of the mounting portion at a non-zero angle.

4. The medical device of claim 1 wherein the outer surface of the mounting portion is threaded around a substantial portion thereof.

5. The medical device of claim 1 further comprising a spacer adapted to engage a superior surface of the distal portion of the main body portion.

6. A medical device, comprising:
a first rod assembly having a main body with an outer surface adapted to support a spacer thereon, a mounting end with a threaded outer surface wherein the main body and the mounting end are substantially aligned; and
a second rod assembly having a main body with an outer surface adapted to support a spacer thereon, a mounting end with a threaded outer surface wherein the main body and the mounting end are substantially aligned.

7. The medical device of claim 6 further comprising a spacer adapted to engage a superior outer surface of the main body of the first rod assembly and a superior outer surface of the main body of the second rod assembly.

8. A method of implanting a device, comprising:
advancing a first cannula having a distal opening into a patient's anatomy toward a first sacral ala of a sacrum;
positioning the distal opening of the first cannula adjacent to the first sacral ala;
inserting into the cannula a rod assembly comprising a main body and a mounting portion;
advancing the rod assembly through the cannula with the mounting portion facing distally;
attaching the rod assembly to the sacral ala;
aligning the main body to be adjacent to a medial sacral crest of the sacrum; and
implanting a spacer in the patient such that an inferior surface of the spacer is mounted on a superior surface of the main body and a superior surface of the spacer is adjacent to a spinous process of the L5 vertebra.

9. The method of claim 8 further comprising adding a bone void filler between the mounting portion and the sacral ala.

10. The method of claim 8 wherein attaching the rod assembly to the sacral ala is accomplished by threading the mounting portion into the sacral ala.

11. The method of claim 8 wherein the advancing the first cannula is performed percutaneously.

12. The method of claim 8 further comprising:
advancing a second cannula into a patient's anatomy toward a second sacral ala of the sacrum;
inserting a second rod assembly comprising a main body and a mounting portion through the second cannula; and
affixing the second rod assembly to the second sacral ala such that the main body of the second rod assembly is adjacent to the medial sacral crest of the sacrum and the main body of the first cannula.

13. The method of claim 12 wherein the advancing the second cannula is performed percutaneously.

14. A method of implanting a device, comprising:
advancing a first cannula having a distal opening into a patient's anatomy toward a first sacral ala of a sacrum in a medial-to-lateral approach;
positioning the distal opening of the first cannula adjacent to the first sacral ala;
inserting into the cannula a first rod assembly comprising a main body and a mounting portion;
advancing the first rod assembly through the cannula;
attaching the first rod assembly to the sacral ala;
aligning the main body of the first rod assembly to be adjacent to a medial sacral crest of the sacrum and inferior to a spinous process of an L5 vertebra;
advancing a second cannula having a distal opening into a patient's anatomy toward a second sacral ala of the sacrum in a medial-to-lateral approach;
positioning the distal opening of the second cannula adjacent to the second sacral ala;
inserting into the second cannula a second rod assembly comprising a main body and a mounting portion;
advancing the second rod assembly through the cannula;
attaching the second rod assembly to the second sacral ala;
aligning the main body of the second rod assembly to be adjacent to the medial sacral crest of the sacrum and the main body of the first rod assembly; and
implanting a spacer in the patient such that an inferior surface of the spacer is mounted on a superior surface of the main body of the first rod assembly and a superior surface of the main body of the second rod assembly.

15. The method of claim 14 further comprising positioning the spacer such that a superior surface of the spacer is aligned with a spinous process of the L5 vertebra.

16. The method of claim 15 further comprising adding a bone void filler between the mounting portion of the first rod assembly and the sacral ala.

17. The method of claim 16 further comprising adding a bone void filler between the mounting portion of the second rod assembly and the second sacral ala.

18. The method of claim 14 wherein the advancing the first cannula is performed percutaneously.

19. The method of claim 18 wherein the advancing the second cannula is performed percutaneously.
